(19) United States
(12) Patent Application Publication
(10) Pub. No.: US 2011/0014246 A1
(43) Pub. Date: Jan. 20, 2011

(54) AMORPHOUS ARFORMOTEROL L-(+)-TARTRATE

(75) Inventors: Girish Dixit, Uttar Pradesh (IN); Nandkumar Gaikwad, Maharashtra (IN); Nitin Sharadchandra Pradhan, Maharashtra (IN); Jon Valgeirsson, Hafnarfjorour (IS)

Correspondence Address: CANTOR COLBURN LLP, 20 Church Street, 22nd Floor, Hartford, CT, 06103, US

(73) Assignee: ACTAVIS GROUP PTC EHF, Hafnarfjörður (IS)

(21) Appl. No.: 12/919,028

(22) PCT Filed: Feb. 27, 2009
(86) PCT No.: PCT/IB2009/005399

§ 371 Date: Oct. 4, 2010

(30) Foreign Application Priority Data
Feb. 28, 2008 (IN) 502/CHE/2008
Sep. 24, 2008 (IN) 2326/CHE/2008

(51) Int. Cl. A61K 31/194; C07C 59/255; A61K 9/00; A61P 11/08

(52) U.S. Cl. 424/400; 562/585; 514/574; 428/402

(57) ABSTRACT

Disclosed herein is a novel and stable amorphous form of arformoterol L-(+)-tartrate, a process for its preparation, pharmaceutical compositions comprising amorphous arformoterol L-(+)-tartrate, and methods of treating with amorphous arformoterol L-(+)-tartrade.

### CROSS REFERENCE TO RELATED APPLICATION

This application claims the benefit of priority to Indian provisional application No. 502/CHE/2008, filed on Feb. 28, 2008; and 2326/CHE/2008, filed on Sep. 24, 2008; which are incorporated herein by reference in their entirety.

### FIELD OF THE DISCLOSURE

Disclosed herein is a novel and stable amorphous form of arformoterol L-(+)-tartrate, process for preparation, pharmaceutical compositions, and method of treating thereof.

### BACKGROUND

U.S. Pat. No. 3,994,974 discloses a variety of α-aminomethylbenzyl alcohol derivatives, processes for their preparation, pharmaceutical compositions comprising the derivatives, and method of use thereof. These compounds have the utility as β-adrenergic stimulants and thus have great activity on respiratory smooth muscle and are suitable as bronchodilating agents. Among them, Formoterol, (±)-N-[2-hydroxy-5-[1-hydroxy-2-[[2-(4-methoxyphenyl)-1-methylethyl]amino]ethyl]phenyl]formamide, is a highly potent and β₂-selective adrenoceptor agonist having a long lasting bronchodilating effect when inhaled. Formoterol is represented by the following structural formula:

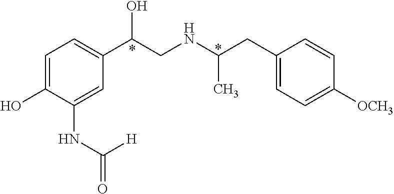

Formoterol has two chiral centers in the molecule, each of which can exist in two possible configurations. This gives rise to four combinations: (R,R), (S,S), (R,S) and (S,R). (R,R) and (S,S) are mirror images of each other and are therefore enantiomers; (R,S) and (S,R) are similarly an enantiomeric pair. The mirror images of (R,R) and (S,S) are not, however, superimposable on (R,S) and (S,R), which are diastereomers. The order of potency of the isomers is (R,R)>>(R,S)=(S,R)>(S,S), and the (R,R)-isomer is 1000-fold more potent than the (S,S)-isomer. Administration of the pure (R,R)-isomer also offers an improved therapeutic ratio.

Various processes for the preparation of Formoterol, its enantiomers and related compounds, and their pharmaceutically acceptable salts are disclosed in U.S. Pat. Nos. 3,994,974; 5,434,304; 6,268,533 and 6,472,563; Chem. Pharm. Bull. 26, 1123-1129 (1978); Chirality 3, 443-450 (1991); Drugs of the Future 2006, 31(11), 944-952; and PCT Publication No. WO 2008/035380A2.

The syntheses of all four isomers of formoterol have been reported in the journals Chem. Pharm. Bull. 26, 1123-1129 (1978) (hereinafter referred to as the ‘CPB Journal’), and Chirality 3, 443-450 (1991) (hereinafter referred to as the ‘Chirality journal’). In the CPB Journal, the (R,R)- and (S,S)-isomers were obtained by diastereomeric crystallization of racemic formoterol with tartaric acid. In the Chirality journal, racemic 4-benzyloxy-3-nitrostyrene oxide was coupled with an optically pure (R,R)— or (S,S)—N-(1-phenylethyl)-N-(1-(p-methoxyphenyl)-2-propyl)amine to give a diastereomeric mixture of formoterol precursors, which were then separated by semipreparative HPLC and transformed to the pure formoterol isomers.

U.S. Pat. No. 6,268,533 discloses that the L-(+)-tartrate salt of R,R-formoterol is unexpectedly superior to other salts of R,R-formoterol (arformoterol), being easy to handle, pharmaceutically innocuous and non-hygroscopic. Arformoterol, N-[2-hydroxy-5-[(1R)-1-hydroxy-2-[[(1R)-2-(4-methoxyphenyl)-1-methylethyl]amino]ethyl]phenyl]formamide, is a highly potent and selective β₂-adrenergic bronchodilator. Arformoterol is represented by the following structural formula 1:

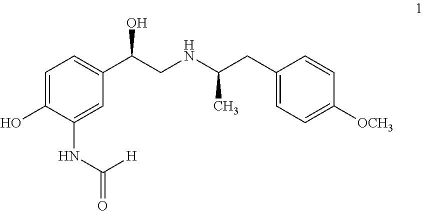

As per the process described in the U.S. Pat. No. 6,268,533 (herein after referred to as the '533 patent), Arformoterol tartrate is prepared by enantioselective reduction of 2-bromo-4′-benzyloxy-3′-nitroacetophenone with borane methyl sulfide in the presence of a chiral oxazaborolidine to produce R-α-(bromomethyl)-4-phenylmethoxy-3-nitrobenzenemethanol, which is then hydrogenated in a Parr hydrogenator in the presence of platinum oxide catalyst to afford the corresponding amino compound followed by formylation reaction with formic acid in the presence of acetic anhydride to produce (R)—N-[5-(2-bromo-1-hydroxyethyl)-2-(phenylmethoxy)phenyl]formamide, which is then treated with potassium carbonate to produce (R)—N-[5-oxiranyl-2-(phenylmethoxy)phenyl]formamide. The epoxide compound is then condensed with (R)-4-methoxy-α-methyl-N-(phenylmethyl)benzene ethanamine L-mandelic acid to produce a dibenzyl protected compound, which is then hydrogenated in the presence of palladium on carbon catalyst to produce arformoterol followed by reaction with L-tartaric acid to produce arformoterol tartrate.

The '533 patent further discloses two crystalline polymorphs (P1 & P2) of R,R-formoterol L-(+)-tartrate salt, and characterizes them by Differential Scanning Calorimetry (DSC). According to the '533 patent, the first polymorph (P1) in pure form exhibits a peak at about 193° C. on differential scanning calorimetry (DSC) and is soluble in water at 25° C. to the extent of 15.4 mg/mL; and the second polymorph (P2) in pure form exhibits a peak at about 179° C. on (DSC) and is soluble in water at 25° C. to the extent of 26.7 mg/mL.

U.S. Pat. No. 6,472,563 (herein after referred to as the '563 patent) discloses a third crystalline polymorph of R,R-formoterol L-(+)-tartrate, designated as “polymorph C”, which is differentiated, from the other two polymorphs (P1 & P2) of R,R-formoterol L-(+)-tartrate which are disclosed in, the '533 patent, by powder X-ray diffraction (P-XRD), DSC, and Infra Red spectroscopy (IR). According to the '563 patent, the polymorphs P1 & P2 of the '533 patent are subsequently denoted as “polymorph A and polymorph B”, the polymorph A is characterized by an X-ray powder diffraction pattern having peaks expressed as 2-theta at about 8.8, 9.3, 12.1, 12.4, 14.2, 15.2, 15.5, 16.8, 18.9, 19.7, 20.8, 22.5, 23.0, 23.7, 25.6, 26.8, 28.6, 30.9, 36.1, 38.1, 39.1, 41.5 and 43.3±0.2 degrees; the polymorph B is characterized by an X-ray powder diffraction pattern having peaks expressed as 2-theta at about 6.7, 7.7, 8.5, 9.9, 11.6, 12.2, 13.0, 13.7, 16.4, 17.3, 19.4, 20.6, 22.1, 22.7, 23.5, 23.9, 24.5, 25.4, 25.5, 26.3, 27.4, 28.6, 29.5, 30.9, 33.0, 37.2, 38.6, 40.9, 41.7 and 44.3±0.2 degrees; and the polymorph C is characterized by an X-ray powder diffraction pattern having peaks expressed as 2-theta at about 6.4, 9.0, 11.1, 12.4, 12.9, 13.5, 14.0, 15.0, 15.4, 15.7, 16.3, 17.5, 19.4, 19.9, 21.3, 22.3, 22.9, 24.1, 24.7, 25.5, 26.0, 26.8, 27.4, 28.4, 29.0, 30.5, 32.7, 34.2, 35.7, 36.4, 37.3, 37.8, 39.3, 39.6, 41.1 and 42.3±0.2 degrees.

Polymorphism is defined as the ability of a substance to exist as two or more crystalline phases that have different arrangement and/or conformations of the molecule in the crystal lattice. Thus, in the strict sense, polymorphs are different crystalline forms of the same pure substance in which the molecules have different arrangements and/or configurations of the molecules. Different polymorphs may differ in their physical properties such as melting point, solubility, X-ray diffraction patterns, and the like. Although these differences disappear once the compound is dissolved, they can appreciably influence the pharmaceutically relevant properties of the solid form, such as handling properties, dissolution rate and stability. Such properties can significantly influence the processing, shelf life, and commercial acceptance of a polymorph. It is therefore important to investigate all solid forms of a pharmaceutical compound, including all polymorphic forms, and to determine the stability, dissolution and flow properties of each polymorphic form. Polymorphic forms of a compound can be distinguished in the laboratory by analytical methods such as X-ray diffraction (XRD), DSC and IR.

Solvent medium and mode of isolation play very important roles in obtaining a polymorphic form over another.

An important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered pharmaceutical compound may reach the patient's bloodstream. The rate of dissolution is a consideration in formulating syrups, elixirs and other liquid medicaments. The solid state form of a compound may also affect its behavior on compaction and its storage stability.

It has been disclosed in the art that the amorphous forms of a number of pharmaceutical compounds exhibit superior dissolution characteristics and in some cases different bioavailability patterns compared to crystalline forms [Konno T., Chem. Pharm. Bull., 38, 2003 (1990)]. For some therapeutic indications one bioavailability pattern may be favored over another.

The discovery of new solid state forms of a pharmaceutical compound provides a new opportunity to improve the performance characteristics of a pharmaceutical product. It enlarges the repertoire of materials that a formulation scientist has available for designing, for example, a pharmaceutical dosage form of a pharmaceutical compound with a targeted release profile or other desired characteristic.

### SUMMARY

The present inventors have now surprisingly and unexpectedly discovered a novel amorphous form of arformoterol L-(+)-tartrate with high purity, adequate stability and good dissolution properties.

The novel amorphous form of arformoterol L-(+)-tartrate is consistently reproducible, does not have the tendency to convert to other forms and found to be more stable even after being stored at a temperature of about 25° C. at a relative humidity of about 55% for at least about 1 month, specifically for a period of 6 months, or at a temperature of about 2-8° C. for at least about 1 month. Moreover, the amorphous form of arformoterol L-(+)-tartrate has a tapped density of greater than about 0.3 g/ml, is less electrostatic, and has good flow properties which is particularly suitable for bulk preparation and handling. The amorphous arformoterol L-(+)-tartrate exhibits properties making it suitable for formulating arformoterol L-(+)-tartrate.

In one aspect, encompassed herein is a process for preparing the highly pure and stable amorphous form of arformoterol L-(+)-tartrate.

In another aspect, an amorphous arformoterol L-(+)-tartrate comprises a water content of less than about 6% by weight based on the total weight of the amorphous arformoterol L-(+)-tartrate.

In another aspect, a pharmaceutical composition comprises amorphous arformoterol L-(+)-tartrate and one or more pharmaceutically acceptable excipients.

In another aspect, a pharmaceutical composition comprises amorphous arformoterol L-(+)-tartrate having a water content of less than about 6% by weight, based on the total weight of the amorphous arformoterol L-(+)-tartrate, and one or more pharmaceutically acceptable excipients.

In still another aspect, a pharmaceutical composition comprises amorphous arformoterol L-(+)-tartrate made by the process disclosed herein, and one or more pharmaceutically acceptable excipients.

In still further aspect, a process for preparing a pharmaceutical formulation comprises combining amorphous arformoterol L-(+)-tartrate with one or more pharmaceutically acceptable excipients.

In another aspect, the amorphous arformoterol L-(+)-tartrate disclosed herein for use in the pharmaceutical compositions has a 90 volume-percent of the particles (D₉₀) having a size of less than or equal to about 300 microns, specifically less than or equal to about 200 microns, more specifically less than or equal to about 100 microns, still more specifically less than or equal to about 70 microns, and most specifically less than or equal to about 15 microns.

### BRIEF DESCRIPTION OF THE DRAWING

FIG. 1 is a characteristic powder X-ray diffraction (XRD) pattern of amorphous arformoterol L-(+)-tartrate.

### DETAILED DESCRIPTION

According to one aspect, there is provided a stable and substantially pure amorphous form of arformoterol L-(+)-tartrate.

According to another aspect, there is provided a stable and substantially pure amorphous arformoterol L-(+)-tartrate having a water content of less than about 6% by weight, based on the total weight of the amorphous arformoterol L-(+)-tartrate.

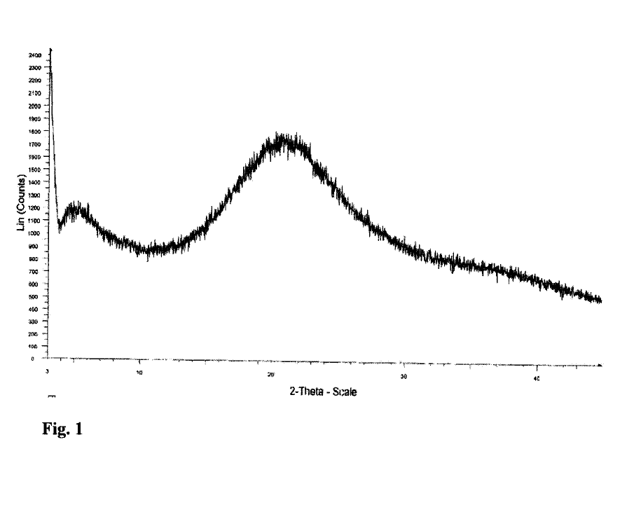

The amorphous form of arformoterol L-(+)-tartrate is characterized by a powder XRD pattern substantially in accordance with FIG. 1. The X-ray powder diffraction pattern shows no peaks, thus demonstrating the amorphous nature of the product.

According to another aspect, a process is provided for the preparation of an amorphous form of arformoterol L-(+)-tartrate, comprising:
a) providing a solution comprising arformoterol L-(+)-tartrate and a solvent, wherein the solvent is an organic solvent or a solvent medium comprising water and an organic solvent;
b) optionally, filtering the solution to remove insoluble matter; and
c) substantially removing the solvent from the solution to provide the amorphous form of arformoterol L-(+)-tartrate.

The term “substantially removing” the solvent refers to at least 30%, specifically greater than about 50%, more specifically greater than about 90%, still more specifically greater than about 99%, and most specifically essentially complete (100%), removal of the solvent from the solvent solution.

The process can produce an amorphous form of arformoterol L-(+)-tartrate in substantially pure form.

The term “substantially pure amorphous arformoterol L-(+)-tartrate” refers to the amorphous arformoterol L-(+)-tartrate having purity greater than about 99%, specifically greater than about 99.5%, more specifically greater than about 99.8% and still more specifically greater than about 99.9% (measured by HPLC).

In an embodiment, the amorphous arformoterol L-(+)-tartrate has a water content of less than about 6.0% by weight, specifically about 0.5-5.5% by weight, and more specifically about 2.5-5.0% by weight, and still more specifically about 2.7-4.8% by weight, based on the total weight of the amorphous arformoterol L-(+)-tartrate.

In another embodiment, the pure amorphous arformoterol L-(+)-tartrate obtained by above process has a water content of about 2.7-4.8% by weight, which is stable and consistently reproducible, and the moisture is retained even after extended drying for 12 hours at about 50-55° C. under vacuum.

In another embodiment, the amorphous arformoterol L-(+)-tartrate remains in the same amorphous form and stable, when stored under nitrogen atmosphere at a temperature of about 25±2° C. and at a relative humidity of about 55±5% for a period of at least one month.

In still another embodiment, the amorphous arformoterol L-(+)-tartrate remains in the same amorphous form and stable, when stored under nitrogen atmosphere at a temperature of about 25±2° C. and at a relative humidity of about 55±5% for a period of 6 months.

In yet another embodiment, the amorphous arformoterol L-(+)-tartrate remains in the same amorphous form and stable, when stored under nitrogen atmosphere at a temperature of about 2-8° C. for a period of at least one month.

In still another embodiment, the amorphous arformoterol L-(+)-tartrate remains in the same amorphous form and stable, when stored under nitrogen atmosphere at a temperature of about 2-8° C. for a period of 6 months.

The term “remains stable”, as defined herein, refers to lack of formation of impurities, while being stored as described hereinbefore.

In another embodiment, the amorphous arformoterol L-(+)-tartrate is a free-flowing solid, having a bulk density of at least about 0.15 g/ml, and specifically about 0.20 g/ml to about 0.26 g/ml.

In another embodiment, the amorphous arformoterol L-(+)-tartrate has a tapped density of at least about 0.26 g/ml, and specifically about 0.30 g/ml to about 0.39 g/ml.

The amorphous arformoterol L-(+)-tartrate obtained by the process disclosed herein is stable, consistently reproducible and has good flow properties, and is particularly suitable for bulk preparation and handling, and hence, the amorphous arformoterol L-(+)-tartrate obtained by the process disclosed herein is suitable for formulating arformoterol L-(+)-tartrate.

The organic solvent used in step-(a) is selected from the group consisting of alcohols, ketones, hydrocarbons, chlorinated hydrocarbons, and mixtures thereof. Specifically, the organic solvent is selected from the group consisting of methanol, ethanol, isopropyl alcohol, n-butanol, tert-butanol, acetone, n-hexane, n-heptane, cyclohexane, toluene, methylene chloride, and mixtures thereof, and more specifically methanol, ethanol, isopropyl alcohol, acetone, and mixtures thereof.

Exemplary alcohol solvents include, but are not limited to, C₁ to C₆ straight or branched chain alcohol solvents such as methanol, ethanol, isopropyl alcohol, n-butanol, tert-butanol, isobutanol, amyl alcohol, and mixtures thereof. Specific alcohol solvents are methanol, ethanol, isopropyl alcohol, and mixtures thereof. Exemplary ketone solvents include, but are not limited to, acetone, methyl ethyl ketone, methyl isobutyl ketone, methyl tert-butyl ketone and the like, and mixtures thereof. A specific ketone solvent is acetone. Exemplary hydrocarbon solvents include, but are not limited to, n-pentane, n-hexane, n-heptane and their isomers, cyclohexane, toluene, xylene, and mixtures thereof. Specific hydrocarbon solvents are n-hexane, n-heptane, cyclohexane, toluene, and mixtures thereof. Exemplary chlorinated hydrocarbon solvents include, but are not limited to, methylene chloride, ethyl dichloride, chloroform, carbontetrachloride, and mixtures thereof. A specific chlorinated hydrocarbon solvent is methylene chloride.

Step-(a) of providing a solution of arformoterol L-(+)-tartrate includes dissolving arformoterol L-(+)-tartrate in the solvent, or obtaining an existing solution from a previous processing step.

In one embodiment, the arformoterol L-(+)-tartrate is dissolved in the solvent at a temperature of below about reflux temperature of the solvent used, specifically at about 20° C. to about 110° C., and still more specifically at about 25° C. to about 80° C.

As used herein, “reflux temperature” means the temperature at which the solvent or solvent system refluxes or boils at atmospheric pressure.

The solution in step-(a) may also be prepared by admixing arformoterol base, L-(+)-tartaric acid and the solvent to obtain a mixture; and stirring the mixture to obtain a solution of arformoterol L-(+)-tartrate. In one embodiment, the mixture is stirred at a temperature of below about reflux temperature of the solvent used for at least 15 minutes, specifically at about 20° C. to about 110° C. from about 20 minutes to about 10 hours, and still more specifically at about 25° C. to about 80° C. from about 30 minutes to about 2 hours.

In another embodiment, the L-(−)-tartaric acid may be used directly or in the form of L-(+)-tartaric acid diluted in a suitable solvent. The solvent used for diluting L-(+)-tartaric acid is selected from the group consisting of water, methanol, ethanol, n-propanol, isopropyl alcohol, n-butanol, isobutanol, tert-butanol, amyl alcohol, acetone, methyl ethyl ketone, methyl isobutyl ketone, methyl tert-butyl ketone, and mixtures thereof.

The solution obtained in step-(a) or step-(b) is optionally subjected to carbon treatment. The carbon treatment is carried out by methods known in the art, for example by stirring the solution with finely powdered carbon at a temperature of below about 70° C. for at least 15 minutes, specifically at a temperature of about 40° C. to about 70° C. for at least 30 minutes; and filtering the resulting mixture through hyflo to remove the carbon to obtain a filtrate containing arformoterol L-(+)-tartrate. In one embodiment, finely powdered carbon is an active carbon.

The solution obtained in step-(a) or step-(b) is optionally stirred at a temperature of about 30° C. to the reflux temperature of the solvent used for at least 20 minutes, and specifically at a temperature of about 40° C. to the reflux temperature of the solvent used from about 30 minutes to about 4 hours.

Removal of solvent in step-(c) is accomplished, for example, by substantially complete evaporation of the solvent, concentrating the solution or distillation of solvent, under inert atmosphere to obtain amorphous arformoterol L-(+)-tartrate.

In one embodiment, the solvent is removed by evaporation. Evaporation can be achieved at sub-zero temperatures by lyophilisation or freeze-drying techniques. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

The distillation process can be performed at atmospheric pressure or reduced pressure. Specifically, the solvent is removed at a pressure of about 760 mm Hg or less, more specifically at about 400 mm Hg or less, still more specifically at about 80 mm Hg or less, and most specifically from about 30 to about 80 mm Hg.

Solvents can also be removed by spray-drying, in which a solution of arformoterol L-(+)-tartrate is sprayed into the spray drier at the flow rate ranging from 10 to 300 ml/hr, specifically 40 to 200 ml/hr. The air inlet temperature to the spray drier used may range from about 30° C. to about 150° C., specifically from about 65° C. to about 110° C. and the outlet air temperature used may range from about 30° C. to about 90° C.

Another suitable method is vertical agitated thin-film drying (or evaporation). Agitated thin film evaporation technology involves separating the volatile component using indirect heat transfer coupled with mechanical agitation of the flowing film under controlled conditions. In vertical agitated thin-film drying (or evaporation) (ATFD-V), the starting solution is fed from the top into a cylindrical space between a centered rotary agitator and an outside heating jacket. The rotor rotation agitates the downside-flowing solution while the heating jacket heats it.

The pure amorphous arformoterol L-(+)-tartrate obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying is carried out at atmospheric pressure or reduced pressures, such as below about 200 mm Hg, or below about 50 mm Hg, at temperatures such as about 25° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The total purity, including the chemical and enantiomeric purity, of the amorphous arformoterol L-(+)-tartrate obtained by the process disclosed herein is greater than about 99%, specifically greater than about 99.5%, more specifically greater than about 99.9%, and most specifically greater than about 99.95% as measured by HPLC. For example, the purity of the amorphous arformoterol L-(+)-tartrate can be about 99% to about 99.95%, or about 99.5% to about 99.99%.

Further encompassed herein is the use of the amorphous form of arformoterol L-(+)-tartrate for the manufacture of a pharmaceutical composition together with a pharmaceutically acceptable carrier.

A specific pharmaceutical composition of amorphous arformoterol L-(+)-tartrate is selected from an aqueous aerosol formulation or a dry powder inhaler composition.

In one embodiment, the amorphous form of arformoterol L-(+)-tartrate has a D₉₀ particle size of less than or equal to about 300 microns, specifically less than or equal to about 200 microns, more specifically less than or equal to about 100 microns, still more specifically less than or equal to about 70 microns, and most specifically less than or equal to about 15 microns.

In another embodiment, the substantially pure amorphous form of arformoterol L-(+)-tartrate disclosed herein for use in the pharmaceutical compositions has a 90 volume-percent of the particles (D₉₀) of less than or equal to about 300 microns, specifically less than or equal to about 200 microns, more specifically less than or equal to about 100 microns, still more specifically less than or equal to about 70 microns, and most specifically less than or equal to about 15 microns.

In another embodiment, the particle sizes of the amorphous form of arformoterol L-(+)-tartrate can be achieved by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for treating a patient suffering from bronchoconstriction or inducing bronchodilation, comprising administering a therapeutically effective amount of the amorphous arformoterol L-(+)-tartrate, or a pharmaceutical composition that comprises a therapeutically effective amount of amorphous arformoterol L-(+)-tartrate, along with pharmaceutically acceptable excipients.

According to another aspect, there is provided pharmaceutical compositions comprising amorphous arformoterol L-(+)-tartrate prepared according to processes disclosed herein and one or more pharmaceutically acceptable excipients.

According to another aspect, there is provided pharmaceutical compositions comprising amorphous arformoterol L-(+)-tartrate having a water content of less than about 6% by weight, based on the total weight of the amorphous arformoterol L-(+)-tartrate prepared according to processes disclosed herein and one or more pharmaceutically acceptable excipients.

According to another aspect, there is provided a process for preparing a pharmaceutical formulation comprising combining amorphous arformoterol L-(+)-tartrate prepared according to processes disclosed herein, with one or more pharmaceutically acceptable excipients.

Yet in another embodiment, pharmaceutical compositions comprise at least a therapeutically effective amount of substantially pure amorphous arformoterol L-(+)-tartrate. Such pharmaceutical compositions may be administered to a mammalian patient in any dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The pure amorphous arformoterol L-(+)-tartrate may also be administered as suppositories, ophthalmic ointments and suspensions, and parenteral suspensions, which are administered by other routes.

The dosage forms may contain substantially pure amorphous arformoterol L-(+)-tartrate as is or, alternatively, may contain substantially pure amorphous arformoterol L-(+)-tartrate as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosages contain substantially pure amorphous arformoterol L-(+)-tartrate within a capsule which may be coated with gelatin. Tablets and powders may also be coated with an enteric coating. The enteric-coated powder forms may have coatings containing at least phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Instrumental Details:

### Purity by HPLC:

### Chromatographic Conditions:

Column: symmetry C18 (150×4.6×5μ)

### Detector: UV at 214 nm

Column temperature: Ambient
Flow rate: 1.0 ml/min
Run time: 60 minutes
Injection volume: 10 μL
Diluent: Water:Acetonitrile—50:50 (% v/v)

### Elution: Gradient

Gradient programme:

| | Mobile phase-A | Mobile phase-B |
|---|---|---|
| Time (min) | (%) | (%) |
| 0 | 84 | 16 |
| 10 | 84 | 16 |
| 37 | 30 | 70 |
| 50 | 30 | 70 |
| 51 | 84 | 16 |
| 60 | 84 | 16 |

| | |
|---|---|
| Buffer solution | Weigh and transfer about 7.5 g of sodium |
| (pH 2.30 ± 0.05) | dihydrogen phosphate in 2000 mL of water |
| | and adjust the pH to 3.00 ± 0.05 with dilute |
| | orthophosphoric acid. Filter through 0.45 μm |
| | or finer porosity membrane and degas |
| Mobile phase-A | Buffer(100% v/v) |
| Mobile phase-B | Acetonitrile |

### X-Ray Powder Diffractometer:

The X-Ray powder diffraction was measured by an X-ray powder diffractometer equipped with a Cu-anode (λ=1.54 Angstrom), X-ray source operated at 40 kV, 40 mA and a Ni filter is used to strip K-beta radiation. Two-theta calibration is performed using an NIST SRM 1976, Corundum standard. The sample was analyzed using the following instrument parameters: measuring range=3-45° 2θ; step width=0.01579°; and measuring time per step=0.11 second.

### Water Content:

The water content was determined by using a Karl Fischer Titrator (Mettler Toledo DL-50 graphics apparatus) according to standard procedures.

### Bulk Density Method of Analysis

About 5 g of sample was placed in a 100 mL measuring cylinder and measured the volume using the following formula:

Bulk density=Weight of the sample (g)/Volume of the sample (mL)

### Tap Density Method of Analysis

About 5 g of sample was placed in a 100 mL measuring cylinder and measured the initial volume. The measuring cylinder was kept in a Sotax Tapping instrument (USP II) and measured the volume of the sample every 50 tappings until a constant volume is obtained using the following formula:

Tap Density=Weight of the sample (g)/constant Volume of the sample after tapping (mL)

### PSD Analysis

Sample was analyzed using Miglyol as a dispersant in Malvern Particle Size instrument with a stirring speed of 3000 rpm.

The following examples are provided to enable one skilled in the art to practice the invention and are merely illustrative of the process of this invention. However, it is not intended in any way to limit the scope of the present invention.

### EXAMPLES

### Example 1

### Preparation of Amorphous Arformoterol L-(+)-tartrate (R,R-formoterol L-(+)-tartrate)

Arformoterol base (5 g), methanol (50 ml) and a solution of L-(+)-tartaric acid (2.18 g) in water (2 ml) were placed in a round bottom flask and the mixture was heated at 60-65° C. under nitrogen atmosphere. The resulting mass was filtered through hyflo bed to get a clear solution, the resulting filtrate was evaporated on rotavapour under reduced pressure at 50-55° C. and the resulting material was further dried under vacuum at 50-55° C. for 18 hours to yield 6.8 g of amorphous arformoterol L-(+)-tartrate (Purity by HPLC: 99.62%; Water content by KF: 3.1% by weight).

### Example 2

### Preparation of Amorphous Arformoterol L-(+)-tartrate

Arformoterol L-(+)-tartrate (10 g) and methanol (550 ml) were placed in a round bottom flask and the mixture was stirred for 30 minutes at 25-30° C. The obtained mass was further heated to 60-65° C. followed by hot filtration through hyflo bed to get a clear solution and the resulting clear filtrate was evaporated on rotavapour under reduced pressure at 50-55° C. and the resulted material was further dried under vacuum at 50-55° C. for 24 hours to yield 11.7 g of amorphous arformoterol L-(+)-tartrate (Purity by HPLC: 99.93%; Water content by KF: 2.77% by weight; Bulk density: 0.239 g/ml; and Tapped density: 0.334 g/ml).

Particle size distribution: d(0.1)=9.78 microns, d(0.5)=31.42 microns, d(0.9)=67.44 microns.

### Example 3

### Preparation of Amorphous Arformoterol L-(+)-tartrate

Arformoterol L-(+)-tartrate (5 g), acetone (250 ml) and methanol (250 ml) were placed in a round bottom flask and the mixture was heated to 60-65° C. followed by hot filtration through hyflo bed to get a clear solution and the resulting clear filtrate was evaporated on rotavapour under reduced pressure at 50-55° C. to yield 4 g of amorphous arformoterol L-(+)-tartrate (Purity by HPLC: 99.68%; Water content by KF: 4.8% by weight).

### Example 4

### Preparation of Amorphous Arformoterol L-(+)-tartrate

Arformoterol base (5 g), methanol (100 ml) and L-(+)-tartaric acid (2.18 g) were placed in a round bottom flask, the resulting mixture was stirred for 2-3 hours at 20-30° C. and further heated at 60-65° C. followed by hot filtration through hyflo bed. The resulting filtrate was evaporated on rotavapour under reduced pressure at 50-55° C. and the resulted material was further dried under vacuum at 50-55° C. for 7 hours to yield 4.2 g of amorphous arformoterol L-(+)-tartrate (Purity by HPLC: 99.8%; Water content by KF: 3.9% by weight).

### Example 5

### Preparation of Amorphous Arformoterol L-(+)-tartrate

Arformoterol L-(+)-tartrate (10 g) and methanol (550 ml) were placed in a round bottom flask and the mixture was stirred for 30 minutes at 25-30° C. The resulting mass was heated at 60-65° C. followed by hot filtration through hyflo bed to get a clear solution. The resulting clear solution was concentrated to dryness using laboratory spray dryer (Jay Instruments & Systems Pvt. Ltd. India, Model-LSD-48 mini Spray Dryer) to give 5.2 g of amorphous arformoterol L-(+)-tartrate (Purity by HPLC: 99.4%).

Conditions of spray drying: Feed rate=20; Inlet temperature=90° C.; Outlet temperature=50° C.; Aspirator=40.

### Example 6

Amorphous arformoterol L-(+)-tartrate (obtained from Examples 1-4) was fine-milled by being passed through a grinder (Make: Morphy Richards, Model-Icon DLX) having a stainless steel liquidizing blade for 3-4 minutes until 90% of the Amorphous arformoterol L-(+)-tartrate had a diameter of less than about 20 microns.

### Example 7

Stability of the Amorphous Arformoterol L-(+)-tartrate

Amorphous arformoterol L-(+)-tartrate samples, having total purity of 99.93% as measured by HPLC, (obtained as per the process described in Example 2) were packed in a low-density polyethylene (LDPE) bag and was sealed using a vacuum-nitrogen sealing machine (purity of nitrogen should be more than 99.99%), which was inserted into a triple laminated aluminum bag (having black inner lining) containing silica and which was then sealed using vacuum-nitrogen sealing machine.

Four samples containing the amorphous arformoterol L-(+)-tartrate, packed in the above-mentioned packaging, were stored at 2-8° C. and at 25±2° C. Samples were withdrawn periodically after 1, 3, 5 and 6 months and the purity of the withdrawn material was checked by HPLC.

All the samples tested conserved the amorphous form after being stored at the specified storage period at the specific temperature. Moreover, the initial total purity of the all the four samples did not change over this period, that is, the purity remained 99.93% after storage.

Unless otherwise indicated, the following definitions are set forth to illustrate and define the meaning and scope of the various terms used to describe the invention herein.

The term “amorphous” means a solid without long-range crystalline order. Amorphous form of arformoterol L-(+)-tartrate specifically contains less than about 10 percent crystalline forms of arformoterol L-(+)-tartrate, more specifically less than 5 percent crystalline forms of arformoterol L-(+)-tartrate, and still more specifically is essentially free of crystalline forms of arformoterol L-(+)-tartrate. “Essentially free of crystalline forms of arformoterol L-(+)-tartrate” means that no crystalline polymorph forms of arformoterol L-(+)-tartrate can be detected within the limits of a powder X-ray diffractometer.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type) is another useful wetting agent, combinations thereof and other such materials known to those of ordinary skill in the art.

As used herein, Dₓ means that X percent of the particles have a diameter less than a specified diameter D. Thus, a D₉₀ or d(0.9) of less than 300 microns means that 90 volume-percent of the particles in a composition have a diameter less than 300 microns.

The term “micronization” used herein means a process or method by which the size of a population of particles is reduced.

As used herein, the term “micron” or “μm” both are same refers to “micrometer” which is 1×10⁻⁶ meter.

As used herein, “Particle Size Distribution (P.S.D)” means the cumulative volume size distribution of equivalent spherical diameters as determined by laser diffraction in Malvern Master Sizer 2000 equipment or its equivalent. “Mean particle size distribution, i.e., D₅₀” correspondingly, means the median of said particle size distribution.

By “substantially pure” is meant having purity greater than about 99%, specifically greater than about 99.5%, and more specifically greater than about 99.9% measured by HPLC.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The term wt % refers to percent by weight. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in, the claims appended hereto as permitted by applicable law. Moreover, any combination of the above-described elements in all possible variations thereof is encompassed by the invention unless otherwise indicated herein or otherwise clearly contradicted by context.

1. Amorphous form of arformoterol L-(+)-tartrate, characterized by a powder XRD pattern in accordance with FIG. 1.

2. (canceled)

3. The amorphous arformoterol L-(+)-tartrate of claim 1, having a purity of about 99% to about 99.99% as measured by HPLC; wherein the amorphous arformoterol L-(+)-tartrate has a water content of less than about 6.0% by weight, based on the total weight of the arformoterol L-(+)-tartrate; wherein the amorphous arformoterol L-(+)-tartrate has a bulk density of at least about 0.15 g/ml, and a tapped density of at least about 0.26 g/ml; and wherein the amorphous arformoterol L-(+)-tartrate has a D₉₀ particle size of less than or equal to about 300 microns.

4. (canceled)

5. The amorphous arformoterol L-(+)-tartrate of claim 3, wherein the amorphous arformoterol L-(+)-tartrate has a water content of about 0.5-5.5% by weight, based on the total weight of the amorphous arformoterol L-(+)-tartrate; wherein the amorphous arformoterol L-(+)-tartrate has a bulk density of about 0.20 g/ml to about 0.26 g/ml, and a tapped density of about 0.30 g/ml to about 0.39 g/ml; and wherein the amorphous arformoterol L-(+)-tartrate has a D₉₀ particle size of less than or equal to about 70 microns.

6. The amorphous arformoterol L-(+)-tartrate of claim 5, wherein the amorphous arformoterol L-(+)-tartrate has a water content of about 2.7-4.8% by weight, based on the total weight of the amorphous arformoterol L-(+)-tartrate.

7. (canceled)

8. (canceled)

9. (canceled)

10. (canceled)

11. (canceled)

12. (canceled)

13. A process for the preparation of amorphous arformoterol L-(+)-tartrate of claim 1, comprising:
a) providing a solution comprising arformoterol L-(+)-tartrate and a solvent, wherein the solvent is an organic solvent or a solvent medium comprising water and an organic solvent, and wherein the organic solvent is selected from the group consisting of alcohols, ketones, hydrocarbons, chlorinated hydrocarbons, and mixtures thereof;
b) optionally, filtering the solution to remove insoluble matter; and
c) substantially removing the solvent from the solution to provide the amorphous form of arformoterol L-(+)-tartrate.

14. (canceled)

15. The process of claim 13, wherein the organic solvent used in step-(a) is selected from the group consisting of methanol, ethanol, isopropyl alcohol, n-butanol, tert-butanol, acetone, n-hexane, n-heptane, cyclohexane, toluene, methylene chloride, and mixtures thereof.

16. (canceled)

17. The process of claim 13, wherein the solution in step-(a) is provided either i) by dissolving the arformoterol L-(+)-tartrate in the solvent at a temperature of below about reflux temperature of the solvent; or ii) by admixing arformoterol base, L-(+)-tartaric acid and the solvent to obtain a mixture; and stirring the mixture to obtain a solution of arformoterol L-(+)-tartrate, wherein the stirring is carried out at a temperature of below about reflux temperature of the solvent used for at least 15 minutes.

18. The process of claim 17, wherein the dissolution is carried out at a temperature of about 20° C. to about 110° C.; and wherein the stirring is carried out at a temperature of about 20° C. to about 110° C. from about 20 minutes to about 10 hours.

19. (canceled)

20. (canceled)

21. (canceled)

22. (canceled)

23. The process of claim 13, wherein the solution obtained in step-(a) is further subjected to carbon treatment; wherein the solution obtained in step-(a) or step-(b) is optionally stirred at a temperature of about 30° C. to the reflux temperature of the solvent used for at least 20 minutes; wherein the removal of the solvent in step-(c) is accomplished by distillation or complete evaporation of the solvent, spray drying, vacuum drying, lyophilization or freeze drying, agitated thin-film (ATFD) drying, or a combination thereof; and wherein the amorphous arformoterol L-(+)-tartrate obtained in step-(c) is further dried under vacuum or at atmospheric pressure, at a temperature of about 25° C. to about 70° C.

24. (canceled)

25. (canceled)

26. (canceled)

27. (canceled)

28. (canceled)

29. (canceled)

30. A pharmaceutical composition comprising amorphous arformoterol L-(+)-tartrate of claim 1 and one or more pharmaceutically acceptable excipients.

31. (canceled)

32. (canceled)

33. The pharmaceutical composition of claim 30, wherein the amorphous arformoterol L-(+)-tartrate has a D₉₀ particle size of less than or equal to about 300 microns.

34. The pharmaceutical composition of claim 33, wherein the amorphous arformoterol L-(+)-tartrate has a D₉₀ particle size of less than or equal to about 70 microns.

35. An aerosol pharmaceutical composition according to claim 30.

36. An oral pharmaceutical composition according to claim 30, wherein the oral pharmaceutical composition is in the form of a tablet, capsule or syrup.

37. (canceled)

38. (canceled)

39. A method of treating a patient suffering from bronchoconstriction or inducing bronchodilation, comprising administering a therapeutically effective amount of amorphous arformoterol L-(+)-tartrate of claim 1, or a pharmaceutical composition that comprises a therapeutically effective amount of the amorphous arformoterol L-(+)-tartrate, along with pharmaceutically acceptable excipients.
